(19) United States
(12) Patent Application Publication
(10) Pub. No.: US 2023/0288368 A1
(43) Pub. Date: Sep. 14, 2023

(54) BIOSENSING ELEMENT, BIOSENSING MODULE, BIOSENSING DEVICE AND BIOSENSING METHOD

(71) Applicant: ACK Biotechnology Co., Ltd., Taichung City (TW)

(72) Inventors: Yi-De LI, Taichung City (TW); Ping-Hua TENG, Taichung City (TW)

(21) Appl. No.: 17/656,651

(22) Filed: Mar. 28, 2022

(30) Foreign Application Priority Data
Mar. 14, 2022 (TW) 111109291
Mar. 14, 2022 (TW) 111109292

(51) Int. Cl. G01N 27/414; G01N 27/12; G01N 27/327; G01N 33/543; G01N 33/49

(52) U.S. Cl. CPC G01N 27/4145; G01N 27/12; G01N 27/3273; G01N 33/5438; G01N 33/49

(57) ABSTRACT

A biosensing element for sensing a target analyte includes a case and a sensing unit. The case has a rod portion and a taper portion, wherein the rod portion is connected to the taper portion. The sensing unit is covered by the case and has a sensing portion exposed from one end of the case by the taper portion. The sensing unit includes a conductor and a reactive material. The conductor has a surface and the reactive material is connected to the surface of the conductor.

### RELATED APPLICATIONS

This application claims priority to Taiwan Application Serial Number 111109291, filed Mar. 14, 2022, and Taiwan Application Serial Number 111109292, filed Mar. 14, 2022, which are herein incorporated by reference.

### BACKGROUND

### Technical Field

The present disclosure relates to a biosensing element, a biosensing module, a biosensing device and a biosensing method. More particularly, the present disclosure relates to a biosensing element, a biosensing module, a biosensing device and a biosensing method which can increase the reaction area and reduce the residual liquid.

### Description of Related Art

Most of the conventional biosensing elements are flat structures, such as biochips, in which samples are taken and dropped onto the flat surface of the sensing element for detection. However, in the above-mentioned method, the samples need to be taken repeatedly, resulting in poor detection efficiency. Further, the sensing surface of the biosensing element is limited and difficult to clean.

A biosensing element with needle structure has already been developed in this field. However, the sample contacts with the sensing element through the collection structure, that is, the sample and the sensing element are not in direct contact. The structure of the above-mentioned biosensing element is complex, and the probability of the occurrence of unstable factors of sensing would be increased.

In addition, most of the biosensing elements are integral structure and cannot be disassembled. The conventional biosensing elements can only sense a single substance or perform a single sensing, which is still limited in use, and the operation thereof is complicated and inconvenient.

Thereof, in order to solve the problem of the conventional biosensing elements, and improve the sensing accuracy thereof, a development of a biosensing element with a three-dimensional structure and a biosensing module with a replaceable biosensing element is a worthy research goal in the related field.

### SUMMARY

According to one aspect of the present disclosure, a biosensing element for sensing a target analyte includes a case and a sensing unit. The case has a rod portion and a taper portion, wherein the rod portion is connected to the taper portion. The sensing unit is covered by the case and has a sensing portion exposed from one end of the case by the taper portion. The sensing unit includes a conductor and a reactive material. The conductor has a surface and the reactive material is connected to the surface of the conductor.

According to another aspect of the present disclosure, a biosensing module includes a plurality of the biosensing elements according to the aspect of the present disclosure and a base. The base has an arranging space. Each of the biosensing elements is detachably disposed in the arranging space of the base, and an arrangement state of the biosensing elements is limited by the arranging space.

According to further another aspect of the present disclosure, a biosensing device for sensing a target analyte includes the biosensing module according to the aspect of the present disclosure and a signal detector. The biosensing elements of the biosensing module react with the target analyte and generate an electrical signal, respectively. The signal detector is electrically connected to the biosensing module and receives the electrical signal.

According to still another aspect of the present disclosure, a biosensing method includes the following steps. The biosensing element according to the aspect of the present disclosure is provided. The biosensing element is used to sense a target analyte, wherein a sensing unit of the biosensing element is contacted with the target analyte directly, and the biosensing element reacts with the target analyte and generates an electrical signal. A signal detector is used to receive the electrical signal and determine a biological characteristic.

According to further still another aspect of the present disclosure includes providing the biosensing module according to the aspect of the present disclosure, performing a sensing step, and performing a replacing step. The biosensing module senses a target analyte of a sample. In the sensing step, the biosensing module is contacted with the sample, and the sensing unit of each of the biosensing elements of the biosensing module reacts with target analyte to sense the target analyte, respectively. In the replacing step, at least one of the biosensing elements is detached from the base, and another of the biosensing element is assembled on the base.

### BRIEF DESCRIPTION OF THE DRAWINGS

The present disclosure can be more fully understood by reading the following detailed description of the embodiment, with reference made to the accompanying drawings as follows:

FIG. 1 is a three dimensional schematic view of a biosensing element according to one example of one embodiment of the present disclosure.

FIG. 2 is an exploded schematic view of the biosensing element of FIG. 1.

FIG. 3 is a sectional schematic view of a sensing unit of the biosensing element of FIG. 1.

FIG. 4 is a partial enlarged view of the biosensing element of FIG. 1.

FIG. 5 is a partial enlarged view of a biosensing element according to another example of one embodiment of the present disclosure.

FIG. 6 is a partial enlarged view of a biosensing element according to further another example of one embodiment of the present disclosure.

FIG. 7 is a schematic view of using the biosensing element of FIG. 1.

FIG. 8 is a flowchart of a biosensing method according to another embodiment of the present disclosure.

FIG. 9 is a three dimensional schematic view of a biosensing module according to one example of further another embodiment of the present disclosure.

FIG. 10 is an exploded schematic view of the biosensing module of FIG. 9.

FIG. 11 is a three dimensional schematic view of a biosensing module according to another example of further another embodiment of the present disclosure.

FIG. 12 is a three dimensional schematic view of a biosensing device according to still another embodiment of the present disclosure.

FIG. 13 is a flowchart of a biosensing method according to still another embodiment of the present disclosure.

### DETAILED DESCRIPTION

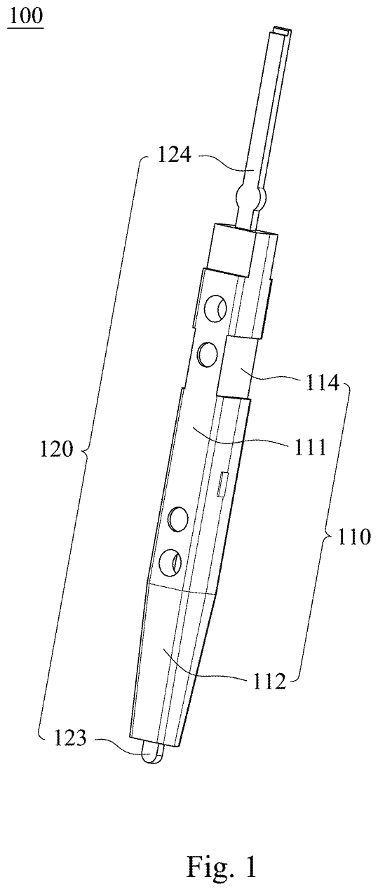

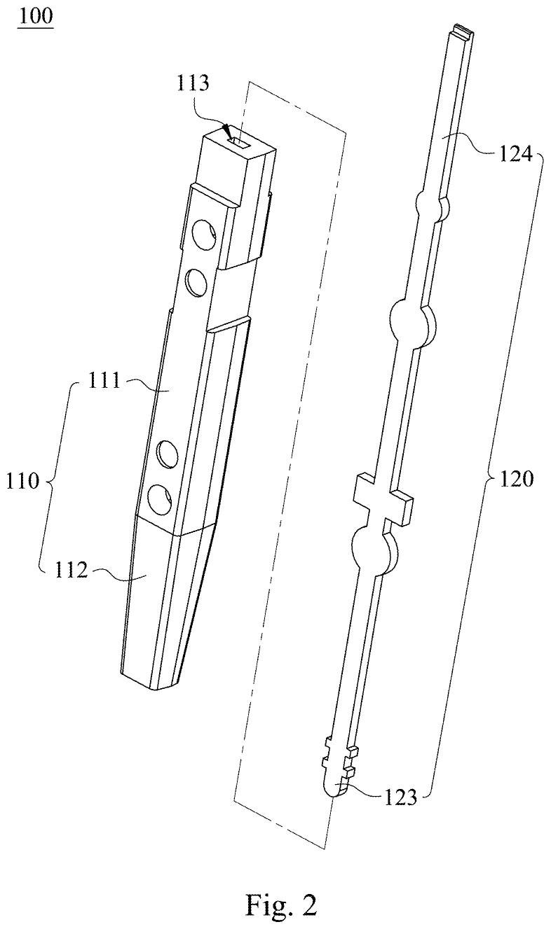

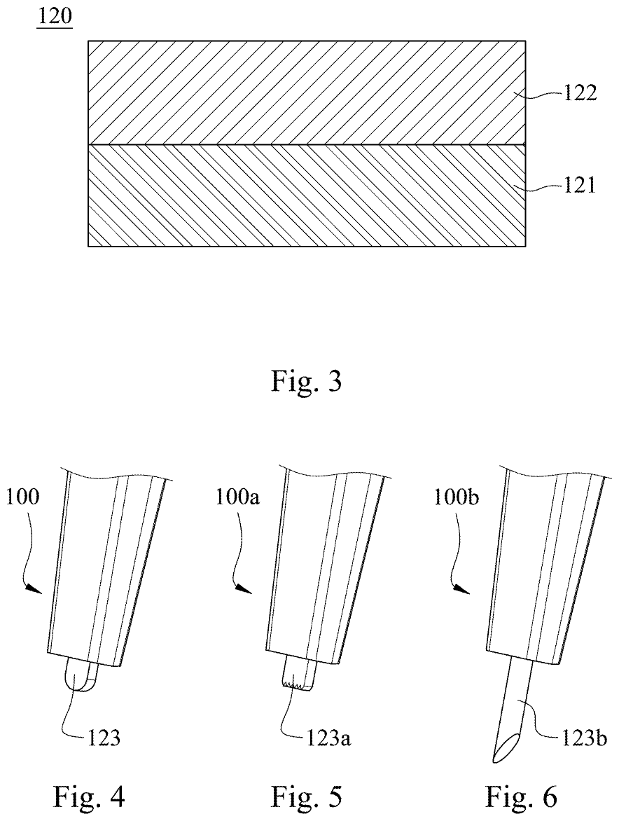

Please refer to FIGS. 1, 2 and 3. FIG. 1 is a three dimensional schematic view of a biosensing element 100 according to one example of one embodiment of the present disclosure. FIG. 2 is an exploded schematic view of the biosensing element 100 of FIG. 1. FIG. 3 is a sectional schematic view of a sensing unit 120 of the biosensing element 100 of FIG. 1. The biosensing element 100 includes a case 110 and a sensing unit 120. The case 110 has a rod portion 111 and a taper portion 112, wherein the rod portion 111 is connected to the taper portion 112. The sensing unit 120 is covered by the case 110 and has a sensing portion 123. The sensing portion 123 is exposed from one end of the case 110 by the taper portion 112. The sensing unit 120 includes a conductor 121 and a reactive material 122. The conductor 121 has a surface and the reactive material 122 is connected to the surface of the conductor 121.

Specifically, the biosensing element 100 is used for sensing a target analyte (not shown). The reactive material 122 can react with the target analyte and generate an electrical signal. The conductor 121 can sense and conduct the electrical signal. In detail, the reactive material 122 contacts and reacts with the target analyte to change the values of current, resistance or impedance in the circuit, thus the biological characteristics can be determined.

The reaction of the reactive material 122 with the target analyte can be an electron transfer triggered by small molecules, oxidation reduction reaction by enzymatic action, and nucleic acid adaptor (Aptamer) to identify the target. For example, the reactive material 122 and the target analyte can be a combination of antibodies and antigens, or the reactive material 122 and the target analyte can be nucleic acids with complementary sequences. The different types of reactive material 122 can be configured according to the types of target analytes to match different reaction mechanisms, and the disclosure will not be limited thereto.

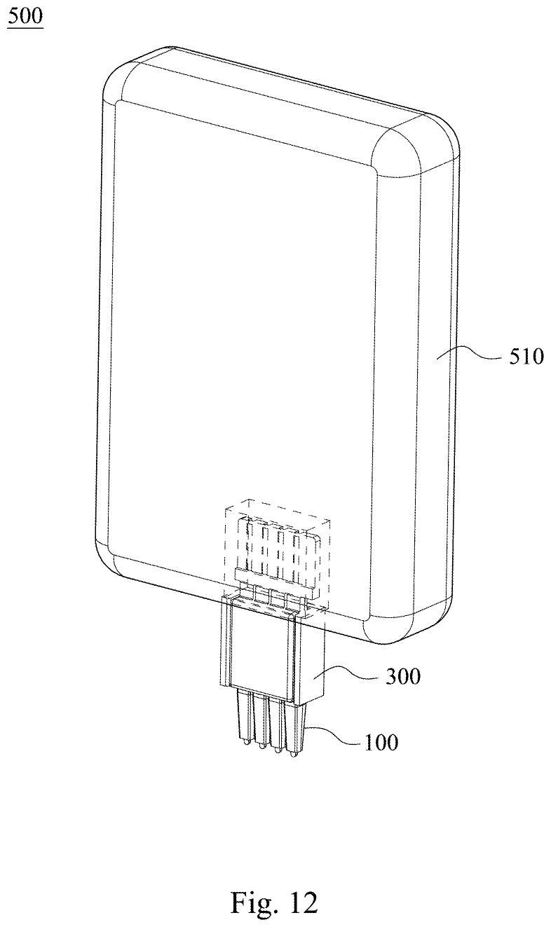

As shown in FIG. 1, the sensing unit 120 has the sensing portion 123 and a conducting portion 124. The sensing portion 123 is exposed from one end of the case 110 by the taper portion 112. The conducting portion 124 is exposed from the other end of the case 110 by the rod portion 111. That is, the sensing portion 123 and the conducting portion 124 are exposed from the two ends of the case 110, respectively. The sensing portion 123 is used for contacting and reacting with the target analyte. The conducting portion 124 is used for connecting an external device (such as the signal detector 510 shown in FIG. 12), so as to transmit the electrical signal to the external device for analysis.

In detail, as shown in FIGS. 1 and 2, the case 110 is a hollow structure and has a passing space 113. The sensing unit 120 is passed through into the passing space 113 of the case 110. In FIG. 1, the four surfaces of the sensing unit 120 are covered by the case 110, and sensing portion 123 and the conducting portion 124 are exposed from the two ends of the case 110, respectively. The biosensing element 100 is a rod-shaped structure, so that at least two surfaces of the sensing portion 123 of the sensing unit 120 can be contacted with the target analyte.

Compared with the conventional sensing element with flat structure, the biosensing element 100 of the present disclosure can increase the contact area between the sensing unit 120 and the target analyte, which can improve the accuracy of the sensing. Further, the reactive material 122 can be disposed on the sensing portion 123 of the sensing unit 120. Therefore, the coating area of the reactive material 122 can be reduced, and the manufacturing cost can be also reduced.

The case 110 can be a non-conductive material. Therefore, the generation and transmission of the electrical signal would not be interfered, and the accuracy of the sensing would not be affected. The case 110 can be made of a corrosion resisting material, so as to effectively protect the sensing unit 120. The case 110 can have a holding portion 114, which is recessed on the surface of the case 110. In detail, the holding portion 114 is recessed on the rod portion 111 of the case 110. The holding portion 114 can be held by the user to facilitate taking the biosensing element 100, and the operation of taking samples can be more convenient.

With the special structural configuration of the biosensing element 100 of the present disclosure, the user can take a sample by the biosensing element 100 directly, wherein the sample includes the target analyte, and the steps of taking the sample to the biosensing element 100 with a micropipette can be omitted. Therefore, the problem of complicated sampling operation of the conventional sensing element with flat structure can be solved. Further, by covering the sensing unit 120 with the case 110, the overall structural strength of the biosensing element 100 can be increased and the risk of deformation or damage to the sensing unit 120 during the sampling process can be avoided.

Please refer to FIGS. 4, 5 and 6. FIG. 4 is a partial enlarged view of the biosensing element 100 of FIG. 1. FIG. 5 is a partial enlarged view of a biosensing element 100a according to another example of one embodiment of the present disclosure. FIG. 6 is a partial enlarged view of a biosensing element 100b according to further another example of one embodiment of the present disclosure. Specifically, the biosensing element 100 of the present disclosure can take the sample directly, and the sensing portion 123 of the sensing unit 120 can be configured into different structures according to requirements, so as to be suitable for various usage situations.

For example, in FIG. 4, one end of the sensing portion 123 of the sensing unit 120 has an arc structure, which can be used to avoid damaging the surface of the sample. In the example of FIG. 5, one end of the sensing portion 123a has a serrated structure, and the sample on the object can be collected by planing or scraping. In the example of FIG. 6, one end of the sensing portion 123b has a needle structure, and the sample can be collected by puncturing the object. Therefore, different structures of the sensing portion can be configured to meet the needs of different sample collection, which can further increase the breadth of application of the biosensing element 100 of the present disclosure.

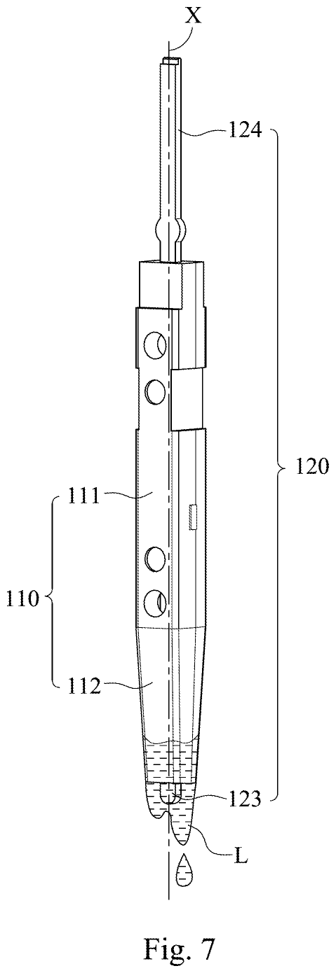

FIG. 7 is a schematic view of using the biosensing element 100 of FIG. 1. Specially, the residual liquid on the biosensing element 100 can be effectively reduced by the structural configuration of the taper portion 112. In detail, as shown in FIG. 7, the taper portion 112 tapers in the direction away from the rod portion 111 along the central axis X of the case 110, so that one end of the case 110 forms a taper structure. Therefore, the liquid L can drip along the taper portion 112. Specifically, the biosensing element 100 can be immersed in a buffering solution before sensing, that is the liquid L shown in FIG. 7, for cleaning and calibration. When the biosensing element 100 is left the buffering solution, the user holds the biosensing element 100 in a vertical direction, and the liquid L drips down due to gravity and concentrates in the taper portion 112. Therefore, the residual liquid L can be effectively avoided, and an effective cleaning effect can be achieved, so as to avoid affecting the sensing, and the sensing accuracy of the biosensing element 100 can be improved.

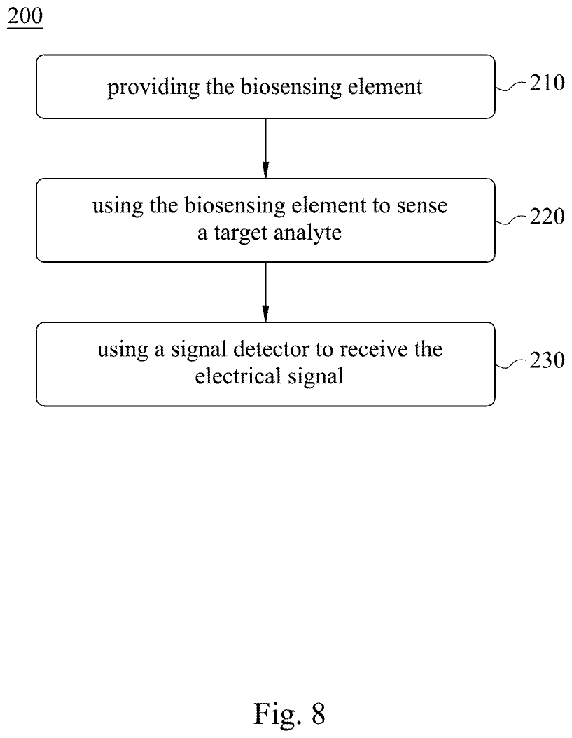

FIG. 8 is a flowchart of a biosensing method 200 according to another embodiment of the present disclosure. The biosensing method 200 includes steps 210, 220 and 230. In step 210, a biosensing element is provided which can be the biosensing element 100 in FIG. 1, but the present disclosure will not be limited thereto. In step 220, the biosensing element senses a target analyte. A sensing unit of the biosensing element is contacted with the target analyte directly, and the biosensing element reacts with the target analyte and generates an electrical signal. In step 230, a signal detector is used to receive the electrical signal and determine a biological characteristic.

In detail, as described in the embodiment of FIG. 1, the biosensing element 100 is a rod-shaped structure, and the overall structural strength of the biosensing element 100 can be increased by the configuration of the case 110. The biosensing element 100 can be used as a collection device to collect samples, and at least two surfaces of the sensing unit 120 can be in direct contact with the target analyte. Therefore, sampling and sensing can be done without the additional tools. Hence, the uncertainties in the sampling process can be reduced, and the convenience of operation of the biosensing method 200 can be improved.

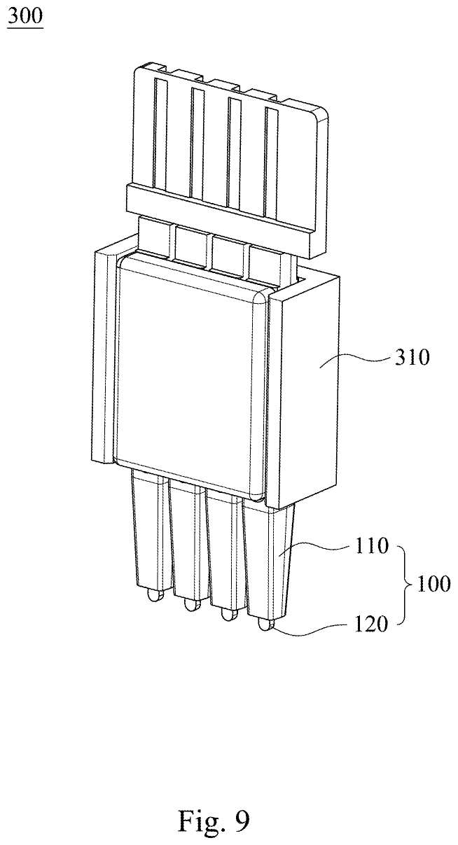

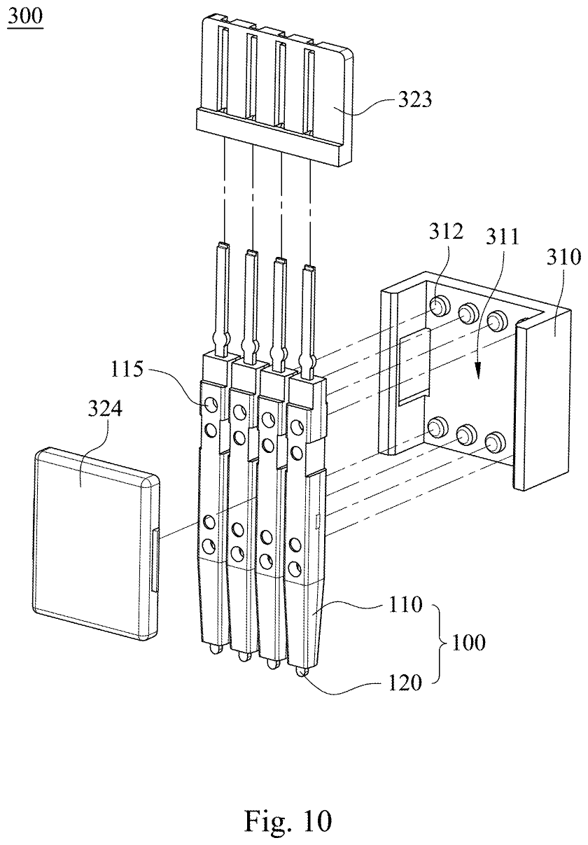

Please refer to FIGS. 9 and 10. FIG. 9 is a three dimensional schematic view of a biosensing module 300 according to one example of further another embodiment of the present disclosure. FIG. 10 is an exploded schematic view of the biosensing module 300 of FIG. 9. The biosensing module 300 includes a plurality of the biosensing elements 100 shown in FIG. 1 and a base 310. The base 310 has an arranging space 311, each of the biosensing elements 100 is detachably disposed in the arranging space 311 of the base 310, and an arrangement state of the biosensing elements 100 is limited by the arranging space 311. The details of the structure of the biosensing element 100 is described in the embodiment of FIG. 1, and will not be described herein again.

Specifically, the reactive material 122 of the sensing unit 120 of one of the biosensing elements 100 is coated with an antigen for detecting the corresponding antibody, and the reactive material 122 of the sensing unit 120 of another one of the biosensing elements 100 is coated with an antibody for detecting the corresponding antigen. Therefore, the operation of simultaneously sensing the antigen and the antibody of the target analyte on the same carrier can be achieved by the biosensing module 300, and the operation can be more convenient and the detection efficiency can be improved.

With the above structure configuration, the biosensing element 100 can be disassembled and replaced from the base 310 according to the usage requirement, so that the biosensing module 300 of the present disclosure can be used more flexibly and more compliant with the needs of customization requirements.

As shown in FIG. 10, the case 110 of each of the biosensing elements 100 can include a first connecting element 115, and the base 310 can include a second connecting element 312. The first connecting element 115 is detachably connected to the second connecting element 312. The first connecting element 115 can be a male piece or a female piece, and the second connecting element 312 can be a male piece or a female piece with a shape corresponding to the first connecting element 115. It should be noted that in other embodiments, the structures and shapes of the first connecting element and the second connecting element can be configured according to the connection requirements, and the present disclosure will not be limited thereto.

It is worth mentioning that the biosensing element 100 is connected to the base 310 through the case 110, rather than directly exerting and acting on the sensing unit 120. Therefore, the sensing unit 120 can be effectively protected by the case 110, and the risk of damage or deformation of the sensing unit 120 during the process of disassembling and replacing the biosensing element 100 can be avoided.

The base 310 can further include a first auxiliary element 323 and a second auxiliary element 324. The first auxiliary element 323 is connected to the sensing unit 120 of each of the biosensing elements 100. The second auxiliary element 324 is connected to the case 110 of each of the biosensing elements 100. In detail, the first auxiliary element 323 is connected to and partially covers the conducting portion 124 of each of the biosensing elements 100. The structural strength of the biosensing elements 100 can be increased to prevent the conducting portion 124 being damaged in the process of connecting to the external device.

One side of the case 110 of each biosensing element 100 is connected to the base 310, the other side of the case 110 of each biosensing element 100 is connected to second auxiliary element 324, and two sides of the second auxiliary element 324 are connected to the base 310. Therefore, with the arrangement of the second auxiliary element 324, the biosensing elements 100 can be stably clamped between the second auxiliary element 324 and the base 310, so that the biosensing elements 100 can be disposed in the arranging space 311 more stably, and the stability of the overall structure of the biosensing module 300 can be increased.

By the special structural configuration of the biosensing element 100 and the structural configuration of the plurality of biosensing elements 100 detachably connected to the base 310, the repeated sampling operation can be omitted, which can be beneficial to improve the efficiency of using the biosensing module 300. Specifically, when a sample is detected, the biosensing module 300 is immersed in the sample, each of the biosensing elements 100 can directly contact and react with its corresponding target analyte at the same time, and each of the biosensing elements 100 generates an electrical signal, respectively. That is, the biosensing module 300 only needs to perform a sampling action once to complete the sensing of each biosensing element 100.

In FIG. 9, a material of the reactive material 122 of each of the biosensing elements 100 can be different from each other, and each of the biosensing elements 100 can sense different kinds of the target analyte. Therefore, the biosensing module 300 can simultaneously sense multiple types of target analytes, so as to improve the operation efficiency.

Further, in the other embodiment, a material of the reactive material 122 of each of the biosensing elements 100 can be the same, and each of the biosensing elements 100 can sense the same kind of the target analyte. Therefore, the biosensing module 300 can generate multiple data for a single target analyte, so as to improve the operation efficiency.

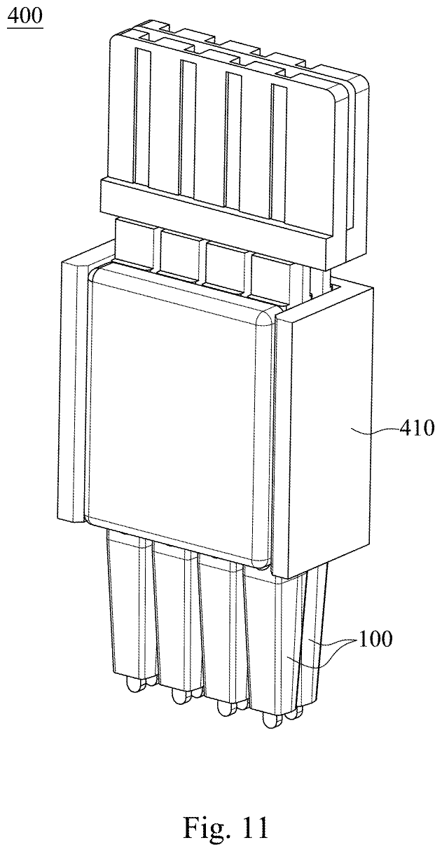

FIG. 11 is a three dimensional schematic view of a biosensing module 400 according to another example of further another embodiment of the present disclosure. The biosensing module 400 includes a plurality of the biosensing elements 100 and a base 410. The structure of the biosensing module 400 in FIG. 11 is similar with the structure of the biosensing module 300 in FIG. 9, and the similarities will not be described herein again. Specifically, in FIG. 11, the number of the biosensing elements 100 is 8, their arrangement state is two arrays.

Further, the arrangement state of the biosensing elements of the biosensing module of the present disclosure can be single column, multi-column, polygon or circle. The biosensing elements can be arranged into various states according to the usage requirement, and the application of the biosensing module of the present disclosure can be more extensive.

FIG. 12 is a three dimensional schematic view of a biosensing device 500 according to still another embodiment of the present disclosure. The biosensing device 500 is used for sensing a target analyte. The biosensing device 500 includes the biosensing module 300 shown in FIG. 9 and a signal detector 510, and the biosensing module 300 includes a plurality of the biosensing elements 100. For the detail of the biosensing element 100 and the biosensing module 300, please refer to the descriptions of FIGS. 1 to 10, and it will not be described herein again.

Specifically, the biosensing elements 100 of the biosensing module 300 react with the target analyte and generate an electrical signal, respectively. The signal detector 510 is electrically connected to the biosensing module 300 and receives the electrical signal to determine the biological characteristics.

With the special structural configuration of the biosensing element 100, the reaction efficiency of the electric current generated with the target analyte can be increased, and the generation of electrical signals can be promoted, which can further enhance the accuracy of the biosensing device 500.

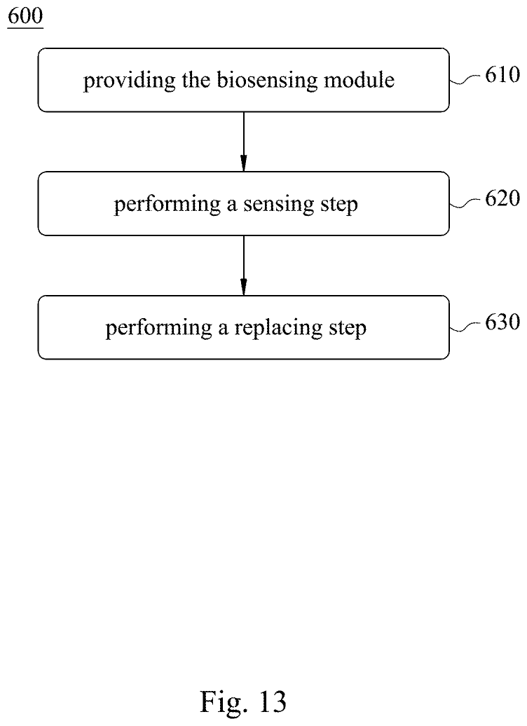

FIG. 13 is a flowchart of a biosensing method 600 according to still another embodiment of the present disclosure. The biosensing method 600 includes steps 610, 620 and 630. Each step will be described in detail below, and the embodiment in FIG. 9 will be used as an illustration for description, but it should be noted that the present disclosure will not be limited thereto.

In step 610, the biosensing module 300 shown in FIG. 9 is provided, wherein the biosensing module 300 senses a target analyte of a sample.

In step 620, a sensing step is performed, wherein the biosensing module 300 is contacted with the sample, and the sensing unit 120 of each of the biosensing elements 100 of the biosensing module 300 reacts with target analyte to sense the target analyte, respectively. In detail, the reactive material 122 of the sensing unit 120 can contact and react with the target analyte to change the values of current, resistance or impedance in the circuit, by sensing the values, an electrical signal is generated. Then, a signal detector is used to receive electrical signal, and through the changes in electrical signal, the biological characteristic can be determined.

Specially, in the sensing step, the reactive material 122 of the sensing unit 120 of one of the biosensing elements 100 is coated with an antigen for detecting the corresponding antibody, and the reactive material 122 of the sensing unit 120 of another one of the biosensing elements 100 is coated with an antibody for detecting the corresponding antigen. The operation of simultaneously sensing the antigen and the antibody of the target analyte on the same carrier can be achieved by the biosensing method 600, and the operation can be more convenient and the detection efficiency can be improved.

In step 630, a replacing step is performed, wherein at least one of the biosensing elements 100 is detached from the base 310, and another of the biosensing element 100 is assembled on the base 310. After completing the sensing step, the replacing step is performed. Through the special structural configuration of the biosensing module 300, the biosensing element 100 can be disassembled and replaced from the base 310 according to the sensing needs, and the operation of the biosensing method 600 can be more convenient and fast.

Specifically, in the replacing step, the biosensing elements 100 can be configured to have different types of the reactive material 122. Therefore, the biosensing method 600 can sense a variety of target analytes, and the types of reactive material 122 can be arbitrarily configured according to different target analytes. Alternatively, the biosensing elements 100 can be configured to have the same type of the reactive material 122. Therefore, the biosensing method 600 can generate multiple data for a single target analyte. By performing the replacing step, the operation of the biosensing method 600 can be more convenient and efficient.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A biosensing element for sensing a target analyte, comprising:
a case having a rod portion and a taper portion, wherein the rod portion is connected to the taper portion; and
a sensing unit covered by the case and having a sensing portion exposed from one end of the case by the taper portion, wherein the sensing unit comprises:
a conductor having a surface; and
a reactive material connected to the surface of the conductor.

2. The biosensing element of claim 1, wherein the reactive material is disposed on the sensing portion of the sensing unit.

3. The biosensing element of claim 1, wherein the case is a non-conductive material.

4. The biosensing element of claim 1, wherein at least two surfaces of the sensing portion of the sensing unit are contacted with the target analyte.

5. The biosensing element of claim 1, wherein one end of the sensing portion has an arc structure, a serrated structure or a needle structure.

6. The biosensing element of claim 1, wherein the sensing unit has a conducting portion, and the conducting portion is exposed from the other end of the case by the rod portion.

7. The biosensing element of claim 1, wherein the case has a holding portion recessed on the surface of the case.

8. A biosensing module, comprising:
a plurality of the biosensing elements of claim 1; and
a base having an arranging space, wherein each of the biosensing elements is detachably disposed in the arranging space of the base, and an arrangement state of the biosensing elements is limited by the arranging space.

9. The biosensing module of claim 8, wherein the case of each of the biosensing elements comprises a first connecting element, the base comprises a second connecting element, the first connecting element is detachably connected to the second connecting element, the first connecting element is a male piece or a female piece, and the second connecting element is a male piece or a female piece with a shape corresponding to the first connecting element.

10. The biosensing module of claim 8, wherein the arrangement state is single column, multi-column, polygon or circle.

11. The biosensing module of claim 8, wherein each of the biosensing elements has a conducting portion which is exposed from the other end of the case by the rod portion, the base comprises a first auxiliary element, and the first auxiliary element is connected to the conducting portion of each of the biosensing elements.

12. The biosensing module of claim 8, wherein a material of the reactive material of each of the biosensing elements is different from each other, so as to sense different kinds of the target analyte.

13. The biosensing module of claim 8, wherein the reactive material of the sensing unit of one of the biosensing elements is coated with an antigen, and the reactive material of the sensing unit of another one of the biosensing elements is coated with an antibody.

14. A biosensing device for sensing a target analyte, comprising:
the biosensing module of claim 8, wherein the biosensing elements of the biosensing module react with the target analyte and generate an electrical signal, respectively; and
a signal detector electrically connected to the biosensing module and receiving the electrical signal.

15. A biosensing method, comprising:
providing the biosensing element of claim 1;
using the biosensing element to sense a target analyte, wherein a sensing unit of the biosensing element is contacted with the target analyte directly, and the biosensing element reacts with the target analyte and generates an electrical signal; and
using a signal detector to receive the electrical signal and determine a biological characteristic.

16. A biosensing method, comprising:
providing the biosensing module of claim 8, wherein the biosensing module senses a target analyte of a sample;
performing a sensing step, wherein the biosensing module is contacted with the sample, and the sensing unit of each of the biosensing elements of the biosensing module reacts with target analyte to sense the target analyte, respectively; and
performing a replacing step, wherein at least one of the biosensing elements is detached from the base, and another of the biosensing element is assembled on the base.

17. The biosensing method of claim 16, wherein in the sensing step, the reactive material of the sensing unit of one of the biosensing elements is coated with an antigen, and the reactive material of the sensing unit of another one of the biosensing elements is coated with an antibody.
